(19) United States
(12) Patent Application Publication
(10) Pub. No.: US 2017/0296453 A1
(43) Pub. Date: Oct. 19, 2017

(54) COSMETIC AND/OR PHARMACEUTICAL COMPOSITION IN THE FORM OF A DISPERSION, METHOD FOR PREPARING SAME AND USE THEREOF FOR SKIN TREATMENT

(71) Applicant: PIERRE FABRE DERMO-COSMETIQUE, BOULOGNE (FR)

(72) Inventors: CATHERINE BIDAN, LABARTHE SUR LEZE (FR); DOMINIQUE CELLIER, FROUZINS (FR); LAURE MOULIS, MONTLAUR (FR); SANDY RATTIER, FLOURENS (FR)

(21) Appl. No.: 15/516,475

(22) PCT Filed: Sep. 30, 2015
(86) PCT No.: PCT/FR2015/052615

§ 371 Date: Apr. 3, 2017

(30) Foreign Application Priority Data
Oct. 2, 2014 (FR) 14 59434

(51) Int. Cl. A61K 8/81; A61K 8/34; A61Q 19/00; A61K 8/37; A61Q 5/12; A61K 8/06; A61K 8/92; A61K 8/31

(52) U.S. Cl. CPC A61K 8/8152; A61K 8/31; A61K 8/342; A61K 8/345; A61Q 19/005; A61K 8/37; A61Q 5/12; A61K 8/062; A61K 8/922; A61Q 19/00; A61K 2800/33; A61K 2800/48; A61K 2800/805; A61K 2800/30

(57) ABSTRACT

A cosmetic, dermatological and/or pharmaceutical composition, in particular for topical use, in the form of a dispersion of a discontinuous internal phase in a continuous external phase. One of the phases is an aqueous phase and the other is an oily phase. The composition contains a polyacrylic polymer and a consistency factor. The composition is free of surfactant and of preservative. A method for preparing the composition includes mixing the aqueous phase and the oily phase so as to form a homogeneous dispersion. The dispersion thus formed is sterilized by ultra-high temperature infusion sterilization.

The present invention falls within the field of the care, protection and/or treatment of the skin, the mucous membranes and/or the hair. More particularly, it relates to a cosmetic, dermatological and/or pharmaceutical composition, in particular intended for topical use, in the form of a dispersion of a discontinuous internal phase in a continuous external phase, that can be used for such care and/or treatment, and also to a method for preparing such a composition.

For reasons linked in particular to their comfort when used, cosmetic, dermatological and/or pharmaceutical compositions intended for topical application are very frequently in the form of emulsions. These emulsions may be of the water-in-oil type, but are usually of oil-in-water type, i.e. comprising an oily phase kept homogeneously dispersed in an aqueous phase using a surfactant.

The cosmetic, dermatological and/or pharmaceutical compositions in the form of emulsions for topical use that are conventionally proposed for the care, protection and/or treatment of the skin comprise numerous ingredients, in particular surfactants intended to stabilize the emulsions, and also preservatives aimed at preserving them against any microbial proliferation for relatively long periods, in multidose packaging. However, such ingredients prove to be irritant to the skin, and in particular to hypersensitive skin, commonly described as intolerant, and/or are capable of causing allergic reactions through their repeated use—the term allergic skin is then used. It thus proves to be desirable to remove them from the makeup of compositions for topical use.

In particular, the skin is covered with a protective film, called hydrolipid surface film. This film constitutes the most external barrier of the skin, and also the most fragile, the most disruptive and the most representative of the health of the skin. It consists to a large extent of the fatty substances excreted by the sebaceous glands and of the lipids originating from cell degradation during the horny cell keratinization phase, and also of hydrophilic compounds, such as water from sweat, glycerol, urea, natural skin moisturization factors, salts, metabolites of the skin flora, etc. This surface film is very exposed and very sensitive to environmental stresses, to personal hygiene routines, and to the condition of the skin. It proves to be important to preserve, and even improve, this barrier function, all the more so for the most sensitive skin. In addition, it is known that populations with intolerant skin of which the skin barrier is weakened require care with physiomimetic hydrolipid agents, in particular physiological emollient and moisturizing agents; it is also important to avoid bringing into contact with the skin any substance capable of degrading the hydrolipid surface film, such as a surfactant or a preservative.

Thus, a method for ultra-high temperature (UHT) infusion sterilization of emulsions for cosmetic, dermatological and/or pharmaceutical use has been provided by the applicant, in patent document WO-A-2013/007755. This sterilization method allows the preservation of such emulsions for a lengthy period, in multidose packaging, without having recourse to preservatives. It also ensures that the integrity of these emulsions is maintained, said emulsions retaining, at the end of the implementation of said method, good homogeneity of dispersion of the internal phase in the external phase.

However, such a sterilization method, which comprises a step of bringing the emulsion into contact with steam at very high temperature, and which may also comprise a step of mechanical treatment by shearing the emulsion, is capable of modifying the rheological properties of the emulsion, in particular of bringing about a modification of its viscosity, and also, more generally, of modifying its organoleptic characteristics, such as its appearance or its texture.

It has now been discovered by the present inventors that it is possible to form a topical composition of the type comprising a discontinuous internal phase homogeneously dispersed in a continuous external phase, which is not only advantageously free of surfactant, but which can also be subjected to such an ultra-high temperature infusion sterilization method, so that it can also be free of any preservative, without degradation of its properties, and even with an improvement in some of them. This is advantageously made possible by a particular choice of the constituents of this composition, more particularly by the combination of a stabilizer of the dispersion of the internal phase in the external phase and of a consistency factor of a particular type. This consistency factor can in particular be chosen from waxy fatty substances, in particular from saturated, preferably C₁₈-C₃₀, hydrocarbons, and in particular from fatty alcohols, such as behenyl alcohol.

After having been subjected to such an ultra-high temperature infusion sterilization method, the composition corresponding to this particular constitution exhibits no loss of its stability nor any degradation of its properties, this being despite the fact that it contains no surfactant. In addition, it has been possible to adjust the viscosity of the composition, and the composition even exhibits improved organoleptic properties, in particular a finer microstructure.

More specifically, the implementation, on a composition having such a particular constitution, of the ultra-high temperature infusion sterilization technique has the effect, entirely surprisingly, of reducing the size of the internal-phase droplets dispersed in the external phase, and also of improving the homogeneity of the dispersion of these droplets in the external phase. Such characteristics give the composition a texture that is particularly pleasant on contact with the skin, and less tacky, and easier and faster to spread than the conventional compositions containing physiomimetic hydrolipid agents.

The present invention thus aims to provide a cosmetic, dermatological and/or pharmaceutical composition, in particular intended for topical application, of the type comprising a discontinuous internal phase homogeneously dispersed in a continuous external phase, which does not contain agents capable of irritating the skin or of generating allergic reactions, and which is compatible with ultra-high temperature infusion sterilization, i.e. which retains its characteristics after such a sterilization, in particular its stability and its viscosity, and even which experiences an improvement in some of its characteristics.

An additional objective of the invention is that this composition is physiocompatible and that it is as close as possible to the original composition of the hydrolipid film of the skin, so as to reinforce the natural surface protection conferred by this film, in particular for hypersensitive skin, with a low degree of tolerance.

To this effect, it is provided according to the present invention a cosmetic, dermatological and/or pharmaceutical composition, in particular for topical use, comprising a discontinuous internal phase dispersed in a continuous external phase, one of these phases, preferably the external phase, being an aqueous phase and the other of these phases, preferably the internal phase, being an oily phase. This composition is in particular intended to be subjected to an ultra-high temperature infusion sterilization method, or has been subjected to such a method.

The composition according to the invention contains a polyacrylic polymer, and also a consistency factor capable of increasing the viscosity of the composition by means of a semi-solid physical thickening phenomenon, which is preferably water-insoluble and liposoluble, i.e. soluble in fatty substances. The composition is also free of surfactant and of preservative.

The polyacrylic polymer is advantageously capable of stabilizing the composition, more particularly the dispersion of the internal phase in the external phase. It is preferably of the type of gelling polymers having hydrophobic zones (such as associative polymers commonly denoted by the expression HASE (Hydrophobic Alkali Swellable Emulsion) polymers. In particular such gelling compounds do not constitute consistency factors as defined by the present invention.

The term “surfactant” is intended to mean, conventionally in itself, any amphiphilic molecule capable of acting on the interfacial tension of a dispersed medium. The composition according to the invention is advantageously free of such surfactant, likely to solubilize the hydrolipid film of the skin.

The composition according to the invention is also devoid of preservatives capable of being responsible for skin intolerances, in particular of any quaternary ammonium, ethanol, phenols, amidines, isothiazolone derivatives, para-hydroxybenzoic esters (known as parabens), etc.

In the present description, the term “preservative” is intended to mean any substance capable of preventing the development of microorganisms in the composition, advantageously by means of its own antimicrobial action. As defined by the present invention, the term “preservative” encompasses both preservatives in the strict sense, in particular as statutorily listed (for example in EC Regulation No. 1223/2009 of the European Parliament and of the Council of 30 Nov. 2009 relating to cosmetic products, definition article 2.1.1 and annex V), and substances called “preservative-like”, not listed by the regulations, but nevertheless having a preservative function. Such substances may be present in cosmetic formulae for other functions (to fragrance, to tone, to firm, etc.), but also have antimicrobial properties, and thus prevent the growth of microorganisms. Thus, excluded from the composition according to the invention, for their preservative effect, are, for example, compounds such as caprylyl glycol, pentylene glycol, ethylhexylglycerol, etc.

Thus, for the purposes of the present invention, the term “preservative” advantageously encompasses the preservatives listed in annex V of EC Regulation No. 1223/2009 of the European Parliament and of the Council of 30 Nov. 2009 relating to cosmetic products, and also caprylyl glycol, pentylene glycol and ethylhexylglycerol.

The composition according to the invention, free of surfactant and of preservative, is in the form of a dispersion of the internal phase in the external phase which, by virtue of the action of the polyacrylic polymer, is homogeneous, i.e. droplets of internal phase are substantially uniformly distributed in the external phase, and stable. In addition, the combination of this polyacrylic polymer with the consistency factor advantageously allows the composition according to the invention to be compatible with an ultra-high temperature infusion sterilization method, i.e. to have, at the end of such a sterilization method, good stability and rheological properties, and more generally organoleptic properties, which are improved.

The term “stability” is intended to mean good homogeneity of dispersion of the internal phase, in the form of microdroplets, in the external phase. A stable composition is in particular a composition which undergoes no release, phase-separation, precipitation, coalescence, flocculation, creaming, etc., phenomena. The composition according to the invention is advantageously such that the equilibrium of the phases of which it is composed is maintained, i.e. the dispersion of the internal phase in the external phase remains homogeneous, after the UHT infusion sterilization method, this being despite the fact that the polyacrylic polymer, the presence of which is recommended by the present invention, is sensitive to shearing. In particular, after the implementation of the sterilization method, including for a long time afterwards, no release, phase-separation, precipitation, coalescence, flocculation, creaming, etc., phenomena occur in the composition according to the invention.

At the end of such a sterilization method, the composition according to the invention may also advantageously be stored for a long period, despite the absence of preservative.

In particular embodiments of the invention, the polyacrylic polymer is a crosslinked copolymer of C₁₀-C₃₀ alkyl acrylate and of acrylic or methacrylic acid, such as the copolymers sold under the names PEMULEN® TR-1 and TR-2, or a blend of such copolymers.

Such a highly crosslinked copolymer has the advantage, compared with the surfactants conventionally used in cosmetic, dermatological and/or pharmaceutical compositions in emulsion form, not only of stabilizing the dispersion of the internal phase in the external phase, but also of gelling the composition. It acts by steric hindrance. It has hydrophilic long chains which form a microgel network around the oil droplets. By virtue of its long-chain (C₁₀-C₃₀) carbon-based groups, this gelled three-dimensional network is kept in place by physical repulsion, which has the effect of stabilizing the oil droplets, and makes it possible to obtain a homogeneous topical composition which is stable over time and which has characteristics close to those of an emulsion, without, however, using surfactants.

In addition, contrary to surfactants, such a copolymer does not penetrate the skin, thereby considerably reducing the risk of it causing an irritation on contact with the skin.

Such a copolymer can in particular be present in the composition in a concentration of between 0.3% and 0.7%, for example between 0.3% and 0.5%, by weight relative to the total weight of the composition.

A consistency factor capable of increasing the viscosity of a composition by means of a semi-solid physical thickening phenomenon is defined in the present invention, conventionally in itself, as a substance that is solid at ambient temperature and has a melting point above 50° C. When incorporated into an oily phase while hot, it returns to its semi-solid consistency when cold and confers on the oily phase a viscosity and a consistency which are regulated by its percentage in the composition.

More particularly, the consistency factor according to the invention is a substance of the water-insoluble and liposoluble type.

In particular excluded from the definition of a consistency factor according to the present invention are gelling agents, in particular water-soluble and/or non-liposoluble gelling agents, such as xanthan gum, sodium polyacrylate, gelling agents such as HASE polymers, etc.

In the composition according to the invention, in combination with the polyacrylic polymer, this consistency factor makes it possible, at the end of the sterilization method, to obtain the viscosity of the composition that is desired. The mechanism underlying the obtaining of such an advantageous effect will not be prejudged here. However, it can be assumed that the physical thickening of the composition containing the polyacrylic polymer by the consistency factor which is soluble in the oily phase, but not in the aqueous phase, contributes to preventing the mechanical stress, induced on the composition by the shearing step of the sterilization method, from breaking the gelled network formed with the internal-phase droplets by the polyacrylic polymer, and thus from modifying the rheology of the composition. The aqueous and oily phases of the composition are also kept intimately linked for long periods, compatible with the lifetime of a cosmetic, dermatological and/or pharmaceutical composition, this being regardless of the respective proportions of each of these phases in the composition.

In addition, the organoleptic properties of the composition are advantageously improved at the end of the sterilization method. In particular, as set out above, the internal-phase microdroplets dispersed in the external phase are finer therein, and dispersed more homogeneously, than in the composition before sterilization.

The consistency factor is preferably chosen from waxy fatty substances, devoid of emulsifying properties, in particular saturated hydrocarbons, preferably comprising a C₁₈-C₃₀ carbon-based chain. Fatty alcohols, for example behenyl alcohol, also called docosanol, having a carbon-based chain comprising 22 carbon atoms, are particularly preferred in the context of the invention. In particular, behenyl alcohol proves to be of particular interest with regard to the physiocompatibility properties of the composition according to the invention. It is indeed a fatty alcohol derived from large fatty acid chains most commonly found in intercorneocyte lipids which participate in the makeup of the upper layers of the epidermis (Stratum corneum).

The consistency factor may in particular be present in the composition in a concentration of between 2% and 7% by weight, relative to the total weight of the composition.

The relative concentrations of the polyacrylic polymer and of the consistency factor in the composition are also chosen so as to obtain, at the end of the sterilization treatment, the desired viscosity for the composition, the consistency factor advantageously making it possible to minimize the consequences, on the viscosity of the composition, of the effect of the ultra-high temperature infusion sterilization treatment on the gelling properties of the polyacrylic polymer.

The composition according to the invention, free of surfactant and of preservative, can be in the form of a homogeneous dispersion of an oily phase in an aqueous phase, of the oil-in-water type. Thus, it can be such that the internal phase is an oily phase and the external phase is an aqueous phase.

Alternatively, it can be in the form of a homogeneous dispersion of an aqueous phase in an oily phase, of the water-in-oil type.

The composition according to the invention can also contain a topically active agent, or a mixture of such active agents.

This active agent can in particular consist of a moisturizing agent, preferably a physiological moisturizing agent, in particular contained in the aqueous phase.

This moisturizing agent, also denoted as physiological humectant, advantageously makes it possible to preserve the surface hydrolipid film and to maintain good surface moisturization of the skin.

By way of such a physiological moisturizing agent which can be a constituent of the composition according to the invention, mention may be made of glycerol (or glycerin), which has in particular the advantages, on the one hand, of being formed naturally during the natural hydrolysis of the triglycerides of the surface hydrolipid film and of the intercorneocyte lipids of the Stratum corneum, and, on the other hand, of having a strong water retention capacity at the skin surface, and therefore a strong capacity for maintaining surface moisturization.

By way of topically active agent, the composition according to the invention can also or otherwise comprise an emollient agent, preferably having a structure close to those found physiologically, such as triglycerides. This emollient agent can in particular be contained in the oily phase.

The proportions of each of this moisturizing agent and of this emollient agent in the composition are conventional in themselves, and can, for example, be between 5% and 10% by weight, relative to the total weight of the composition, for the moisturizing agent, in particular glycerol, and between 5% and 12% by weight, relative to the total weight of the composition, for the emollient agent, in particular caprylic/capric acid triglycerides.

The aqueous phase and the oily phase are, for their part, used in relative proportions that are customary in the cosmetics, dermatological or pharmaceutical field, according to the desired galenical form for the composition. The determination of these relative proportions is within the competence of those skilled in the art.

According to a particularly advantageous characteristic of the invention, the composition preferably comprises a physiological moisturizing agent, contained in the aqueous phase, and a physiological emollient agent, contained in the oily phase.

The composition according to the invention, corresponding to such a characteristic, advantageously participates, when it is applied to the skin surface, in a surface reequilibration of the natural lipids and humectants of the surface hydrolipid film.

In addition, slightly deeper, in the upper layers of the epidermis, it makes it possible to fill the intercorneocyte spaces with suitable lipids without causing occlusion. This is in particular due to the very fine nature of the oily-phase microdroplets making up the emulsion, in particular at the end of the ultra-high temperature infusion sterilization method, which allows easier penetration of these microdroplets between the corneocytes, and faster and more efficient filling of the protective lipid lamellae of the skin. The corneocyte lipid filling is accordingly all the more persistent.

The composition according to the invention preferably contains less than 10 ingredients in total.

Advantageously, none of these ingredients is of the type with a risk of intolerance, or of the type which modifies the biology of the skin. They are preferably all physiocompatible ingredients, so that the composition according to the invention can then be described as a physiocomposition.

In particular, the composition according to the invention is preferentially devoid of fragrance, of dye, or else of antimicrobial, bactericidal or bacteriostatic, fungistatic or fungicidal agent, so that it respects the equilibrium of the skin. It is preferably also devoid, among the usual adjuvants for cosmetic, dermatological and/or pharmaceutical compositions, of glycols (other than glycerol), of silicones, of mineral oils, of lanolin and its derivatives, of antioxidants of BHT (butylated hydroxytoluene) or BHA (butylated hydroxyanisole) type, of chelating agents such as EDTA (ethylenediaminetetraacetic acid), etc.

The choice of the ingredients which are constituents of the oily phase is also preferentially limited to the ingredients which, in addition to the fact that they are cosmetically, dermatologically and/or pharmaceutically compatible, also have a similarity with the lipids of the surface hydrolipid film and of the Stratum Corneum, and are not occlusive.

Thus, the oily phase preferably contains one or more ingredients chosen from fatty materials of vegetable origin, for their content in physiological fatty acids and triglycerides, such as shea butter, rich in omega-6 and omega-9 fatty acids, and safflower oil, rich in omega-6 fatty acids, squalane, for its composition mimicking the sebum of the surface hydrolipid film, medium-chain triglycerides for their presence in the natural state in the surface hydrolipid film and their low oxidation potential, etc.

The composition according to the invention can in particular be in a topically applicable form.

For example, it can be in the galenical form of a milk, a cream, a mask or a fluid.

The composition according to the invention, in cream form, can for example correspond to the following constitution, the amount of each of the ingredients being expressed as percentage by weight, relative to the total weight of the composition:

Aqueous Phase

| | |
|---|---|
| Demineralized or spring water | 64% to 74% |
| Glycerol | 5% to 10% |

Oily Phase

| | |
|---|---|
| Safflower oil | 5% to 10% |
| Hydrogenated shea butter | 0% to 10% |
| Caprylic/capric acid triglycerides | 5% to 12% |
| Behenyl alcohol | 2% to 7% |
| C₁₀-C₃₀ acrylate polymer | 0.3% to 0.7%. |

Preferentially, the composition according to the invention is sterile. The sterility of the composition is defined here in a manner conventional in itself, in accordance with Standard NF EN 556 and with the European pharmacopoeia in force, as the probability of a microorganism proliferating in the composition. Typically, this probability, for a sterile product, is below 10⁻⁶.

The composition according to the invention preferably has a level of sterility as obtained for a sterilizing value F0=22 minutes. Such a level of sterility can in particular be obtained by subjecting the composition according to the invention to an ultra-high temperature infusion sterilization treatment as described in document WO-A-2013/007755.

The sterilizing value of the sterilization method which makes it possible to achieve the desired level of sterility for the composition, F0, the method of determination of which is defined by the European pharmacopoeia in force, corresponds to a time, expressed in minutes, quantifying the lethal effect of humid heat at 121° C. on viable microorganisms. The lethal effect is measured in relation to a reference germ, sporulated Geobacillus stearothermophilus. This germ is particularly resistant and tolerant to heat. The value of F0 is given by the formula:

F0=t.10^{(T-121/z)}

where t is the treatment time expressed in minutes,
z has the dimension of a temperature and is defined by the heat resistance of the microorganism under consideration. The value of z is defined experimentally in comparison with a parameter D. D is a decimal reduction time and measures the time, at a given temperature, here 121° C., to reduce the concentration of the microorganism under consideration by 90%. For Geobacillus stearothermophilus, D is equal to 1 min. Thus, z is the variation in temperature which modifies the value of D by a factor of 10. For Geobacillus stearothermophilus, z is equal to 10° C. These factors D and z depend on the medium and vary in particular according to the type of composition,
T is the treatment temperature.

Thus, a treatment with a sterilizing value F0 equal to 22 minutes is a treatment for 22 minutes at 121° C. (394 K), or else a treatment for 36 seconds at 135° C. (408 K).

The composition according to the invention may thus be obtained by means of method comprising, after a step of dispersion of the internal phase in the external phase, by mixing the aqueous phase and the oily phase, each containing the appropriate ingredients, a step of ultra-high temperature infusion sterilization, as described hereinafter.

According to another subject, the present invention thus also relates to a method for preparing a composition according to the invention, having one or more of the characteristics described above. This method comprises steps of:
preparing the aqueous phase and the oily phase, the oily phase containing the consistency factor, and at least one of the aqueous phase and the oily phase, for example the oily phase, containing the polyacrylic polymer,
mixing the aqueous phase and the oily phase, so as to form a composition in which one of the phases is homogeneously dispersed in the other phase, and
where appropriate, sterilizing the composition thus formed, by high temperature infusion.

According to particular embodiments of the invention, the step of sterilizing the composition by high temperature infusion comprises the steps consisting in:

a. gradually preheating the composition to a composition stability limit temperature, called preheating temperature, in particular of 55° C.;

b. performing an ultra-high temperature infusion sterilization of the composition thus preheated, comprising:
i. heating to a sterilization temperature, in particular of 145° C.,
ii. maintaining at the sterilization temperature, in particular for at least 6 s,
iii. cooling under vacuum to an end-of-sterilization temperature, in particular of 50° C.,

c. gradually cooling with stirring to a storage temperature, for example of approximately 30° C., in particular in steps, the difference in temperature between two steps preferably being less than or equal to 15° C.

The preheating and the cooling steps are preferentially carried out by means of scraped-surface heat exchangers, as described in patent document WO-A-2013/007755.

In particular embodiments of the invention, the sterilization step also comprises a mechanical treatment step, termed mixing step, by shearing of the composition after the ultra-high temperature sterilization step b.

It can also comprise a step of mixing the composition after step c of gradual cooling to the storage temperature.

Another aspect of the invention relates to the use of a composition according to the invention, corresponding to one or more of the above characteristics, for the cosmetic treatment of the skin, the mucous membranes and/or the hair.

To this effect, the composition according to the invention can be topically applied to the skin, the mucous membranes and/or hair.

Thus, a method for cosmetic treatment of the skin, the mucous membranes and/or the hair comprises the topical application, to the skin, the mucous membranes and/or the hair, of a composition according to the invention.

The present invention relates, moreover, to the use of a composition according to the invention for the curative and/or preventive treatment of diseases of the skin, of the mucous membranes and/or of the hair.

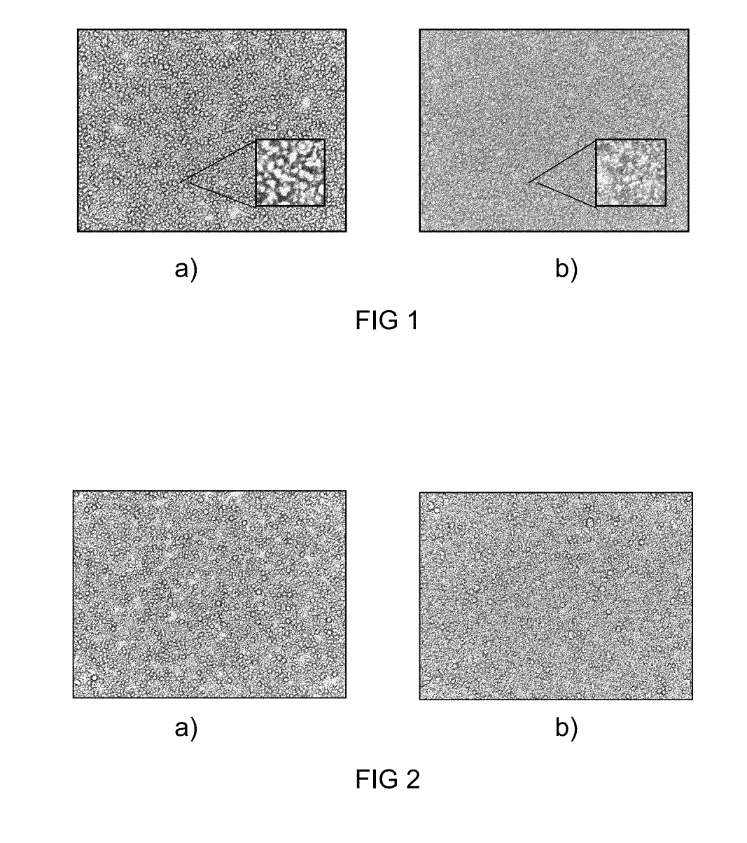

The characteristics and advantages of the invention will emerge more clearly in the light of the examples of implementation hereinafter, which are provided simply by way of illustration and in no way limiting the invention, with the support of FIGS. 1 and 2, in which:

FIG. 1 shows images obtained under a microscope, with a 40-times magnification, of a composition in accordance with the present invention in the form of a cream, a) before and b) after a high-temperature infusion sterilization treatment; and

FIG. 2 shows images obtained under a microscope, with a 40-times magnification, of a composition in accordance with the present invention in the form of a milk, a) before and b) after a high-temperature infusion sterilization treatment.

### EXAMPLE 1

A cream in accordance with the invention, particularly suitable for hypersensitive and allergic skin, is prepared by mixing the phases below. For each constituent, the percentage by weight, relative to the total weight of the composition, is indicated.

Aqueous Phase

| Demineralized or spring water | qs 100% |
|---|---|
| Glycerol | 5%-10% |

The mixture is brought to 40° C.

Oily Phase

| | |
|---|---|
| Safflower oil | 5%-10% |
| Hydrogenated shea butter | 5%-10% |
| Caprylic/capric acid triglycerides | 5%-12% |
| Behenyl alcohol | 2%-7% |

The mixture is brought to 80° C., then the following ingredient is added:

| | |
|---|---|
| PEMULEN ® TR-2 | 0.3%-0.7%. |

The oily phase is gradually introduced into the aqueous phase, so as to homogeneously disperse it in the latter, and then the mixture is cooled to 28° C., and filtered.

At the end of these steps, a composition is obtained in which fine droplets of oily phase are relatively homogeneously dispersed in the aqueous phase. The viscosity of this composition is measured at 11800 cps.

The composition thus obtained is subjected to a high-temperature infusion sterilization treatment, according to the method described in document WO-A-2013/007755, by means of a sterilization device as described in said document, comprising an ultra-high temperature (UHT) infusion sterilization device, scraped-surface heat exchangers for preheating and cooling the emulsion, and means for mechanical treatment by shearing the composition at the outlet of the UHT sterilization device. The operating parameters are as follows:

a. gradually preheating the composition to a preheating temperature of 55° C.;

b. infusion-sterilizing the composition thus preheated, comprising:
i. heating to a sterilization temperature of 145° C.,
ii. maintaining this sterilization temperature for 6 s,
iii. cooling under vacuum to an end-of-sterilization temperature of 50° C.,

c. gradually cooling with stirring to a storage temperature of 30° C., via a step at 40° C.

The frequency of the heat exchangers is 50 Hz.

At the end of this sterilization method, the appearance of the composition is evaluated. This appearance is very vividly white and shiny. The feeling on the skin is that of a light and fine cream, which is easy to spread and which rapidly penetrates into the skin. The viscosity is measured at 7054 cps.

The composition is observed under a microscope (Leica ICC50HD microscope), with a 40-times magnification. The image obtained is shown in FIG. 1 (in b)). By way of comparison, the image obtained by observation under a microscope, at the same magnification, of the composition before the high temperature infusion sterilization treatment is also shown in this figure, in a). It is clearly observed therein, in particular on the magnified partial view, that the oily-phase droplets dispersed in the aqueous phase are smaller in size after the sterilization treatment. These droplets are also dispersed more homogeneously in the aqueous phase.

The size of these droplets was evaluated by means of a graduated slide fitted on the microscope, before and after the sterilization treatment. The values obtained are the following: before the sterilization treatment, the droplet size is between 5 and 8 □m; after the sterilization treatment, the droplet size is between 2 and 3 □m. The size of the oily-phase droplets dispersed in the aqueous phase is thus much smaller, and what is more with a narrower size distribution, after UHT infusion sterilization, than before such a sterilization.

The sterilization method thus not only made it possible to do away with the addition of preservatives in the composition, but it also induced an improvement in the organoleptic properties of this composition.

### EXAMPLE 2

A makeup-removing milk in accordance with the invention, particularly suitable for hypersensitive and allergic skin, is prepared as described in Example 1 above, with the difference that the aqueous and oily phases have the following composition (for each constituent, the percentage by weight, relative to the total weight of the composition is indicated):

Aqueous Phase

| | |
|---|---|
| Demineralized or spring water | 77% to 79% |
| Glycerol | 2% to 10% |

Oily Phase

| | |
|---|---|
| Squalane | 5% to 10% |
| Caprylic/capric acid triglycerides | 5% to 12% |
| Behenyl alcohol | 2% to 7% |
| C₁₀-C₃₀ acrylate polymer | 0.3% to 0.5% |

At the end of the sterilization method, a composition is obtained in which very fine droplets of oily phase are very homogeneously dispersed in the aqueous phase.

The composition is observed under a microscope, with a 40-times magnification. The image obtained is shown in FIG. 2 (in b)). By way of comparison, the image obtained by observation under a microscope, at the same magnification, of the composition before the high temperature infusion sterilization treatment is also shown in this figure, in a). It is clearly observed therein that the size of the oily-phase droplets dispersed in the aqueous phase is smaller after the sterilization treatment. The droplets are also more homogeneously dispersed therein.

### EXAMPLE 3

A cream in accordance with the invention, particularly suitable for hypersensitive and allergic skin, is prepared by mixing the phases below. For each constituent, the percentage by weight, relative to the total weight of the composition, is indicated.

Aqueous Phase

| Demineralized or spring water | qs 100% |
|---|---|
| Glycerol | 5%-10% |
| PEMULEN ® TR-2 | 0.3%-0.7% |

Oily Phase

| | |
|---|---|
| Safflower oil | 5%-10% |
| Hydrogenated shea butter | 5%-10% |
| Caprylic/capric acid triglycerides | 5%-12% |
| Behenyl alcohol | 2%-7%. |

The composition thus obtained is subjected to a high temperature infusion sterilization treatment, according to the method described in Example 1 above.

1-17. (canceled)

18. A cosmetic, dermatological or pharmaceutical composition, comprising a discontinuous internal phase dispersed in a continuous external phase, one of the phases being an aqueous phase and other of the phases being an oily phase, the composition comprising a polyacrylic polymer and a consistency factor capable of increasing a viscosity of the composition utilizing a semi-solid physical thickener, which is water-insoluble and liposoluble, and the composition being free of surfactant and of preservative.

19. The composition according to claim 18, wherein the polyacrylic polymer is a crosslinked copolymer of C₁₀-C₃₀ alkyl acrylate and of acrylic or methacrylic acid.

20. The composition according to claim 18, wherein the consistency factor is chosen from waxy fatty substances.

21. the composition according to claim 20, wherein the consistency factor is chosen from saturated hydrocarbons.

22. The composition according to claim 21, wherein the consistency factor is chosen from saturated C₁₈-C₃₀ hydrocarbons.

23. The composition according to claim 18, wherein the internal phase is an oily phase and the external phase is an aqueous phase.

24. The composition according to claim 18, further comprising at least one topically active agent.

25. The composition according to claim 24, further comprising a moisturizing agent.

26. The composition according to claim 24, further comprising an emollient agent.

27. The composition according to claim 18 is sterile according to Standard EN 556 and European pharmacopoeia.

28. The composition according to claim 27 is sterile according to the Standard EN 556 and the European pharmacopoeia, with a level of stability as obtained for a sterilizing value F0=22 minutes.

29. The composition according to claim 18 contains less than 10 ingredients in total.

30. The composition according to claim 18 is in a topically applicable form.

31. The composition according to claim 18 is in a form of a milk, a cream, a mask or a fluid.

32. A method for preparing the composition of claim 18, comprising steps of:
preparing the aqueous phase and the oily phase, the oily phase contains the consistency factor, and at least one of the aqueous phase and the oily phase contains the polyacrylic polymer; and
mixing the aqueous phase and the oily phase to form the composition in which one of the phases is homogeneously dispersed in the other of the phases.

33. The method according to claim 32, further comprising, after the step of mixing the aqueous phase and the oily phase, a step of sterilizing the composition formed by high temperature infusion.

34. The method according to claim 33, wherein the step of sterilizing the composition comprises steps of:
gradually preheating the composition to an emulsion stability limit temperature;
performing an ultra-high temperature infusion sterilization of the composition thus preheated, by: heating to a sterilization temperature, maintaining at the sterilization temperature, and cooling under vacuum to an end-of-sterilization temperature; and
gradually cooling with stirring to a storage temperature.

35. A method of cosmetic treatment of a skin, of mucous membranes or hair, using the composition of claim 18.

36. The method of according to claim 35, further comprising a step of topically applying the composition to the skin of the mucous membranes or the hair.

37. A method of curative or preventive treatment of diseases of the skin, of the mucous membranes or of the hair, using the composition of claim 18.
